(19) United States
(12) Patent Application Publication
(10) Pub. No.: US 2004/0138757 A1
(43) Pub. Date: Jul. 15, 2004

(54) Eccentric neck for femoral hip prosthesis

(76) Inventors: Mark E. Nadzadi, Austin, TX (US); Eric J. Schantz, Austin, TX (US)

Correspondence Address: ZIMMER TECHNOLOGY, INC., 150 N. WACKER DRIVE, SUITE 1200, CHICAGO, IL, 60606, US

(21) Appl. No.: 10/339,215

(22) Filed: Jan. 9, 2003

(51) Int. Cl. A61F 2/32

(52) U.S. Cl. 623/22.11

(57) ABSTRACT

An implantable femoral hip stem having an offset neck extending outwardly from the proximal region of the stem. The neck has two parallel axes that are separated by an offset distance. A first axis is concentric with the distal end of the neck, and a second axis is concentric with the proximal end of the neck. Together, the two axes form an eccentric neck.

### FIELD OF THE INVENTION

The disclosure herein relates generally to implantable orthopedic prostheses and, more particularly, to an offset neck for a femoral hip prosthesis.

### BACKGROUND OF THE INVENTION

In the United States alone, over 160,000 hip replacements are performed each year. Degenerative arthritis, or the gradual degeneration of the hip joint, is the most common reason for these replacements. Unfortunately, patients can experience problems with the prosthetic hip after a total hip replacement surgery.

Dislocation of the prosthetic hip is one problem patients can experience. The percentage of patients who experience dislocation varies. The rate of dislocation for primary total hip replacements, for example, is relatively low at around 5%. By contrast, the rate of dislocation for hip revisions is much higher; some studies have reported rates of 10% or more.

In light of the large number of hip replacements each year, much scientific research has been done to study the phenomenon of dislocation. Researchers have discovered that hip dislocation actually occurs in several different stages. In the first stage, an initial or slight degree of impingement originates with the bone or the prosthesis. In a second more advanced stage, subluxation occurs. Subluxation represents incomplete dislocation of the prosthesis or the point between impingement and total dislocation of the femoral head from the acetabulum. Finally, in a third stage, the femoral head dislodges from the acetabulum.

A large amount of research has been directed toward reducing the rate of impingement and dislocation. Researchers have addressed important questions in this area: What causes dislocation, and what modifications can be made to prevent its occurrence?

Today, researchers generally agree that a host of factors contribute to impingement and dislocation. These factors include the type and skill of surgical approach employed, the design of prosthetic components, postoperative care and management, and the position or orientation of the prosthetic component once implanted.

Regarding this latter factor, an important correlation exists between the occurrence of impingement and the position in which the hip prosthesis is implanted in the patient. The abduction angle of the acetabular cup is one factor affecting this correlation. Researchers have discovered that an acetabular cup with an abduction angle between 40°-50° can decrease impingement. The degrees of femoral and acetabular anteversion are other factors affecting impingement. A low femoral anteversion has been linked to increase occurrence of impingement and relevant reductions in range of motion. Preferably, the femoral component is inserted in about 10°-20° of anteversion.

Recently, much research has been devoted to understand the correlation between the occurrence of impingement and the size of the femoral head. Scientific studies with cadavers have shown that larger femoral heads can significantly improve the overall range of motion of a prosthetic hip prior to impingement and subluxation.

Simply installing a larger femoral head, though, will not necessary guarantee a reduction in impingement or dislocation. Other factors must be taken into account as well. Researchers have discovered, for example, that the design of the neck of the prosthesis plays an important role in improving range of motion and reducing impingement and dislocation. As such, studies have examined to what extend longer neck lengths coupled with enlarged femoral heads can increase range of motion and reduce impingement. Such studies have concluded that a longer neck length can offer distinct advantages when coupled with an enlarged head.

Other research has been directed toward the head and neck as well, and numerous questions have been posed in this area. How does the ratio of sizes of both the femoral head and neck affect range of motion? To what extent can offsetting the head from the neck increase range of motion?

This latter question has been addressed, and a consensus exists that offsetting the femoral head from the neck provides beneficial increases in range of motion and decreases in impingement and dislocation. To realize this offset, many designs have been proposed to offset the femoral head from the neck or implant body. U.S. Pat. No. 5,910,171, for example, teaches a head having a mounting means that is off-center or not concentric with the central axis of the head. Other designs have utilized complex multi-piece modular femoral stem components to achieve this offset.

It therefore would be advantageous to provide an implantable femoral hip prosthesis with an offset neck to increase range of motion and joint stability, decrease the occurrence of impingement and dislocation, and provide other benefits and advantages.

### SUMMARY OF THE INVENTION

The present invention is directed to a femoral hip prosthesis having an offset or eccentric neck. The hip prosthesis has an elongated body or stem extending from a proximal region to a distal region. A longitudinal axis extends through the body. The proximal region or trochanteral section has an outwardly projecting neck that extends from a distal end connected to the proximal region to a proximal end connected to a femoral ball.

The configuration of the neck is the important and novel feature of the present invention. Two separate axes extend through the neck. A first axis or neck axis is concentric with the distal end of the neck, and a second axis or trunion axis is concentric with the proximal end of the neck. These axes are parallel to each other, extend through the body of the neck, and form an acute angle with the longitudinal axis of the body of the hip prosthesis. Together, the two axes form an eccentric neck with the center of the distal end offset from the center of the proximal end.

The eccentric neck enables the femoral head to be offset. In other words, the femoral head is concentric with trunion axis while being offset from the distal end of the neck and the neck axis. Preferably, the neck axis is superior to the trunion axis to provide the noted offset.

The neck can be formed integrally with the body of the hip and extend outwardly from the proximal region. Alternatively, the neck can be formed as a separate, modular piece. In this latter configuration, a modular connection exists between the distal end of the neck and the proximal region of the hip. A taper may be formed on the distal end of the neck to engage and connect with a corresponding tapered recess on the proximal region of the hip.

One important advantage of the present invention is that the eccentric neck provides an increase range of motion to the femoral hip prosthesis. This increase in range of motion more fully emulates the anatomical movements of a natural hip. Additionally, this increase in range of motion provides more joint stability to the implanted prosthesis.

Another important advantage of the present invention is that the eccentric neck provides a femoral hip prosthesis that is less likely to experience impingement, subluxation, and ultimately dislocation.

### BRIEF DESCRIPTION OF THE DRAWINGS

The invention will now be explained in more detail with reference to the drawings, wherein:

FIG. 1 is a side view of a femoral hip prosthesis of the present invention connectable to a femoral head.

FIG. 2 is a perspective view of the femoral hip prosthesis and femoral head of FIG. 1.

FIG. 3 is a perspective view of the femoral hip prosthesis and femoral head of FIG. 1 connectable to an acetabular component.

FIG. 4 is an exploded side view of an alternate embodiment of the present invention.

FIG. 5 is a perspective view of the embodiment of FIG. 4.

### DETAILED DESCRIPTION

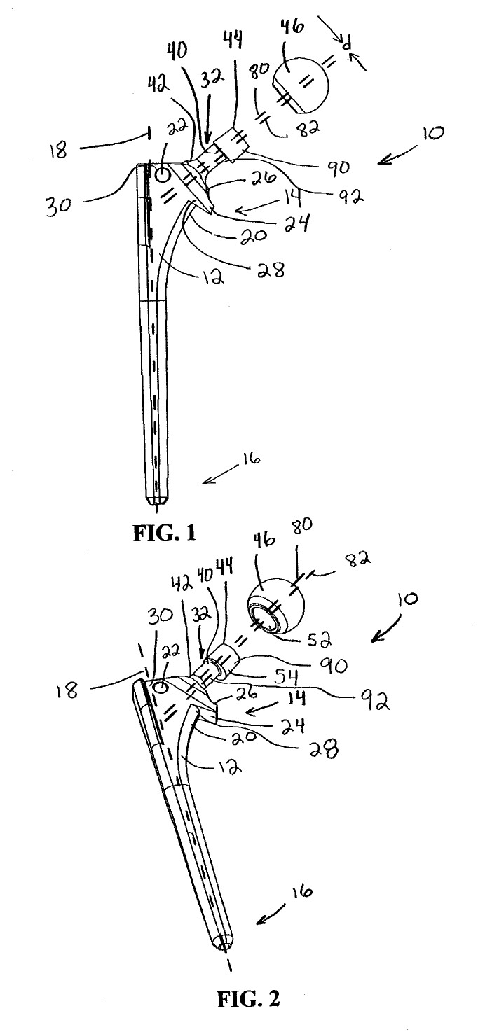

Looking to FIGS. 1-3, an implantable orthopedic femoral hip implant 10 is shown. Implant 10 includes a body 12 that extends from a proximal region 14 to a distal region 16. The body tapers downwardly and generally has a cylindrical or trapezoidal shape with the distal end being rounded to facilitate insertion into the intramedullary canal of a femur. A longitudinal axis 18 extends through the body.

The proximal region 14 includes a proximal body portion or trochanteral portion 20 having an optional cylindrical bore 22 and a collar 24. A top surface 26 extends generally between a lesser trochanter portion 28 to a greater trochanter portion 30. A neck 32 extends outwardly from the top surface 26.

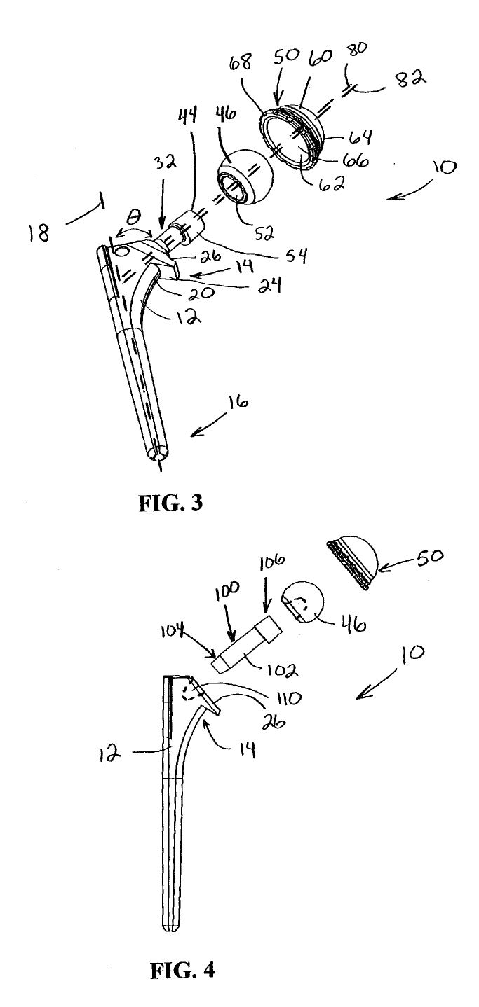

The neck 32 has a body 40 with a distal end 42 that connects to the top surface 26 and a proximal end 44 that connects to a femoral head or ball 46. The ball has a spherical configuration with an outer surface adapted to engage with an acetabular component 50 (FIG. 3). A tapered female recess 52 extending into the ball is shaped to receive a tapered portion 54 of the proximal end 44 of the neck 32. The two tapers can be conFigured to press-fit together in a Morse taper connection.

The acetabular component 50 is conFigured to fit in the acetabulum of a patient and is formed from an outer shell 60 and an inner liner or bearing component 62. The shell is generally shaped as a hemispherical cup defined by an outer hemispherical surface or bone engaging surface 64 and an inner hemispherical surface 66 connected to the bearing component. The inner and outer surfaces define a shell wall having an annular rim 68. The outer surface can be porous or textured while the inner surface is smooth and adapted to articulate with the femoral head 46.

One skilled in the art will appreciate that the novel features of the present invention can be employed with various implants and implant designs without departing from the scope of the invention. The hip implant 10, for example, can be the Apollo® Hip or Natural™ Hip manufactured by Centerpulse Orthopedics Inc. of Austin, Tex.; and the acetabular component 50 can be the Allofit™ or Converge™ acetabular system manufactured by the same company.

Turning now to a more detailed examination of the neck, an important and novel feature of the present invention. Two separate axes extend through the body 40 of neck 32. A first axis or neck axis 80 is concentric with the distal end 42 of the neck, and a second axis or trunion axis 82 is concentric with the proximal end 44 of the neck. In the preferred embodiment, these two axes are parallel to each other and form an acute angle 0 (FIG. 3) with the longitudinal axis 18 of the body 12 of the hip prosthesis 10. The axes are eccentric with the longitudinal axis; in other words, they do not have or share a common center with the longitudinal axis. The axes are also distinct from each other and are separated by an offset distance “d” (FIG. 1). The offset distance, preferably, ranges from about 0.1 mm to 5 mm. Together, the two axes form an eccentric neck.

The eccentric neck 32 enables the femoral head 46 to be offset. In other words, the femoral head 46 is concentric with trunion axis 82 while being offset from the distal end 42 of the neck and the neck axis 80. Preferably, the neck axis 80 is superior to the trunion axis 82 to provide the noted offset.

In the preferred embodiment, the neck 32 is formed as one-piece and is integrally formed with the body 12 of the hip and extends outwardly from the proximal region 14 and, in particular, surface 26. The neck has an elongated cylindrical or conical configuration with a first cylindrical or conical portion 90 and a second, larger cylindrical or conical portion 92 (FIGS. 1 and 2). The first portion has a diameter that ranges from about 10 mm to 14 mm; and the second portion has a diameter that ranges from about 9 mm to 18 mm. Preferably, portion 92 tapers toward its end.

Although FIGS. 1-3 show the neck axis 80 parallel to the trunion axis 82, these axes do not have to be parallel to provide an offset. The trunion axis, for example, could be canted with respect to the neck axis. In this scenario, an offset could still be provided between the proximal and distal ends of the neck. Further, the two axes do not have to be straight. These axes, for example, can be curvilinear or straight small segments put together to form non-linear axes.

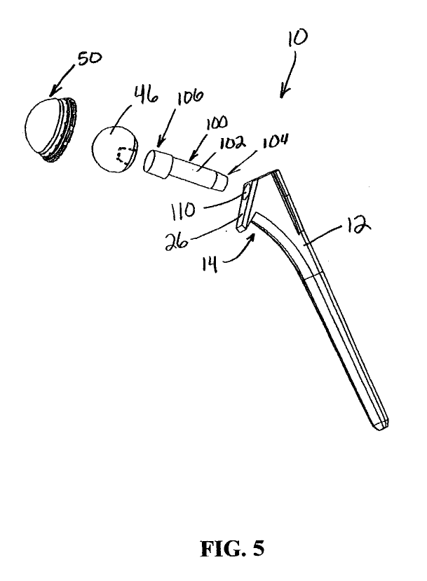

Looking now to FIGS. 4 and 5, an alternate embodiment of the present invention is shown. These Figures generally show an implant 10, a femoral ball 46, and an acetabular component 50 as described in connection with FIGS. 1-3, wherein like numerals are used for all Figures. One important difference is that eccentric neck 100 is not integrally formed with body 12 of hip prosthesis 10. Instead, the neck is formed as a separate, modular component that is removeably connectable to the implant as shown.

The neck 100 has a body 102 with a distal end 104 that connects to the top surface 26 and a proximal end 106 that connects to a femoral head or ball 46. Distal end 104 is tapered to matingly engage in a Morse taper with a correspondingly sized tapered recess 110 extending into the proximal region 14 from top surface 26 of hip implant 10. This taper connection enables the neck to be rotated to an infinite number of positions before the distal end of the neck is locked with the hip implant. Further, the taper connection enables the proximal end of the neck to be rotated to an infinite number of positions with the femoral head before these components are locked together. The neck has two axes and an offset as described in FIGS. 1-3.

FIGS. 4 and 5 illustrate that the proximal and distal ends of the neck can connect to the hip implant and femoral ball, respectively, with a taper connection. One skilled in the art will realize that various types of connections could also be employed and still remain within the scope of the invention. These connections include, but are limited to, press-fit connections, locking rings, radial or expandable devices (such as sleeves or collars), nitinol or other superelastic materials, taper connections, locking connections, various polygonal connections (such as triangular, square, hexagonal, or trapezoidal), and the like. Further, the Figures illustrate that the femoral ball is connectable to the proximal end of the neck. The femoral ball, however, can also be integrally formed to the proximal end of the neck.

One important advantage of the present invention is that the eccentric neck provides an increase range of motion to the femoral hip prosthesis. This increase in range of motion offers the patient a wider, safer range of flexibility and more joint stability. Further, a hip prosthesis with the eccentric neck of the present invention more fully emulates the anatomical movements of a natural hip and decreases the likelihood and occurrence of impingement, subluxation, and ultimately dislocation.

The following chart summarizes a comparison between the range of motion of a standard femoral hip prosthesis (specifically, the Apollo hip manufactured by Centerpulse Orthopedics Inc. of Austin, Tex.) versus a hip prosthesis with an eccentric neck of the present invention. A full range of motion for both hip prostheses was conducted. The first column (“Position”) shows the various positions in 22.5° increments of the leg from flexion, to adduction, to extension, and finally to abduction and back to flexion. The second column (“Standard Neck”) illustrates the degree of movement in the particular position for the standard femoral hip prosthesis. The third column (“Eccentric Neck”) shows the corresponding degree of movement for the hip prosthesis with the eccentric neck. Finally, the last column (“Difference”) illustrates the difference in degrees between the standard neck and eccentric neck.

| | Standard | Eccentric | |
|---|---|---|---|
| Position | Neck | Neck | Difference |
| Flexion | 112.0 | 120.0 | 8.0 |
| FFAD | 94.6 | 101.7 | 7.1 |
| FAD | 75.1 | 81.8 | 6.7 |
| FADAD | 64.4 | 70.3 | 6.0 |
| Adduction | 52.1 | 58.5 | 6.4 |
| EADAD | 45.6 | 51.0 | 5.4 |
| EAD | 44.5 | 50.5 | 6.0 |
| EEAD | 46.0 | 53.4 | 7.4 |
| Extension | 50.7 | 57.6 | 6.9 |
| EEAB | 54.5 | 58.3 | 3.8 |
| EAB | 54.7 | 57.0 | 2.3 |
| EABAB | 57.4 | 59.5 | 2.1 |
| Abduction | 65.1 | 65.3 | 0.2 |
| FABAB | 75.4 | 74.8 | −0.6 |
| FAB | 82.3 | 83.5 | 1.1 |
| FFAB | 99.2 | 100.9 | 1.8 |
| Endorotation | 145.4 | 156.4 | 11.0 |
| Exorotation | 71.8 | 73.4 | 1.6 |

As shown in the chart, the eccentric neck of the present invention offered, in many positions, significant improvement of more than 5° over the standard neck. In fact, the improvement was as high as 8° in flexion and 11° for endorotation. In only one position (FABAB) was the difference negative, being −0.6°. Further, it should be noted that the software used to obtain the data was validated to be accurate to ±1 degree.

Although illustrative embodiments have been shown and described, a wide range of modifications, changes, and substitutions is contemplated in the foregoing disclosure and in some instances, some features of the embodiments may be employed without a corresponding use of other features. Accordingly, it is appropriate that the appended claims be construed broadly and in a manner consistent with the scope of the embodiments disclosed herein.

What is claimed is:

1. An implantable femoral hip prosthesis, comprising:
a stem being adapted to be inserted into an intramedullary canal of a patient, having an elongated body extending from a proximal region to a distal region, and having a longitudinal axis extending through the body; and
a neck projecting outwardly at an acute angle to the longitudinal axis and extending from a distal end that is connected to the proximal region of the body to a proximal end that is adapted to be connected to a femoral head, wherein the neck has two separate and parallel axes that extend from the proximal to distal ends, a first axis being concentric with the distal end, and a second axis being concentric with the proximal end.

2. The implantable femoral hip prosthesis of claim 1 in which the neck is formed as one-piece and integrally formed to the proximal region of the body.

3. The implantable femoral hip prosthesis of claim 2 in which the first and second axes are separated by a distance ranging from about 0.1 mm to 5 mm.

4. The implantable femoral hip prosthesis of claim 3 in which the first and second axes form an acute angle with the longitudinal axis.

5. The implantable femoral hip prosthesis of claim 1 in which the neck is formed as one-piece and the first and second axes are offset from one another.

6. The implantable femoral hip prosthesis of claim 5 in which the neck has an elongated configuration with a first conical portion at the distal end and a second conical portion at the proximal end, wherein the second conical portion is larger than the first conical portion.

7. The implantable femoral hip prosthesis of claim 6 in which the first conical portion has a diameter that ranges from about 10 mm to 14 mm, and the second conical portion has a diameter that ranges from about 9 mm to 18 mm.

8. An implantable femoral hip prosthesis, comprising:
a stem extending from a proximal region to a distal region, and having a longitudinal axis extending through the stem; and
a separate, modular neck extending from a distal end that is adapted to be connected to the proximal region of the stem to a proximal end that is adapted to be connected to a femoral head, the neck having a first axis that is concentric with the distal end and a second axis that is concentric with the proximal end, wherein the first and second axes are offset from one another and form axes that are different than the longitudinal axis.

9. The implantable femoral hip prosthesis of claim 8 in which the first and second axes form an acute angle with the longitudinal axis.

10. The implantable femoral hip prosthesis of claim 8 in which the first and second axes are parallel.

11. The implantable femoral hip prosthesis of claim 8 in which the first and second axes are offset by a distance ranging from about 0.1 mm to 5 mm.

12. The implantable femoral hip prosthesis of claim 11 in which the neck has a first cylindrical portion at the distal end and a second cylindrical portion at the proximal end, wherein the second cylindrical portion is larger than the first cylindrical portion.

13. The implantable femoral hip prosthesis of claim 12 in which the proximal and distal ends are tapered.

14. The implantable femoral hip prosthesis of claim 8 in which the distal end of the neck is removeably connectable to the proximal region of the stem.

15. The implantable femoral hip prosthesis of claim 14 in which the neck is formed as one-piece.

16. An implantable femoral hip prosthesis, comprising:
an stem being adapted to be inserted into an intramedullary canal of a patient, having a body extending from a proximal region to a distal region, and having a longitudinal axis extending through the body; and
a neck projecting outwardly at an acute angle to the longitudinal axis and extending from a proximal end to a distal end that is connected to the proximal region of the body, wherein the neck has two separate and distinct axes, a first axis being concentric with the distal end, and a second axis being concentric with the proximal end, both the first and second axes being eccentric with the longitudinal axis.

17. The implantable femoral hip prosthesis of claim 16 in which the proximal end is offset from the distal end.

18. The implantable femoral hip prosthesis of claim 17 in which the offset ranges from about 0.1 mm to 5 mm.

19. The implantable femoral hip prosthesis of claim 17 in which the neck is integrally formed with the stem.

20. The implantable femoral hip prosthesis of claim 17 in which the neck is removeably connectable to the stem.
